(19) United States
(12) Patent Application Publication
(10) Pub. No.: US 2011/0257702 A1
(43) Pub. Date: Oct. 20, 2011

(54) SELF-ASSEMBLED MONOLAYER COATING ON ELECTRICALLY CONDUCTIVE REGIONS OF A MEDICAL IMPLANT

(76) Inventors: Sule Kara, New South Wales (AU); James F. Patrick, New South Wales (AU); Martin Svehla, New South Wales (AU)

(21) Appl. No.: 13/062,443

(22) PCT Filed: Sep. 4, 2009
(86) PCT No.: PCT/AU2009/001158

§ 371 Date: Jun. 29, 2011

(30) Foreign Application Priority Data
Sep. 4, 2008 (AU) 2008904592

(51) Int. Cl. A61N 1/00; A61F 2/18

(52) U.S. Cl. 607/55; 623/10

(57) ABSTRACT

A medical implant having an electrically conductive region for stimulating tissue of a user or patient. The electrically conductive region is coated with a self assembled monolayer (SAM) which at least inhibits the attachment of impedance-inducing material such as protein, cells or fibrous tissue, to the electrically conductive region.

### CROSS-REFERENCE TO RELATED APPLICATIONS

The present application is a national stage application of International Application No. PCT/AU2009/001158, filed Sep. 4, 2009, and claims the benefit of Australian Provisional Patent Application No. 2008904592, filed Sep. 4, 2008. The content of these applications are hereby incorporated by reference herein.

### BACKGROUND

1. Field of the Invention

The present invention relates to medical implants and, more particularly, to a self-assembled monolayer coating on electrically conductive regions of a medical implant.

2. Related Art

A variety of medical implants deliver electrical energy to tissue of a patient to stimulate that tissue. Examples of such implants include pace makers, auditory brain stem implants (ABI), Functional Electrical Stimulators (FES), Spinal Cord Stimulators, cochlear implants etc.

In a cochlear implant, an array of electrodes are implanted into the patient's cochlea. The electrode array is connected to a stimulator unit that is operationally connected to a signal processing unit. In operation, audio signals from the environment are received from a microphone and delivered to the signal processing unit for audio processing. These processed signals are used by the stimulator unit to generate electrical signals for delivery to the cochlea via the electrode array. As such, a cochlear implant allows for electrical signals to be applied directly to the auditory nerve fibers of a patient, thereby allowing the brain to perceive a sound.

The generation and delivery of the electrical stimulating signals requires energy provided by, for example, a battery. Energy drain of such a power source can severely affect the effectiveness and performance of a medical implant. As would be appreciated, the drain of energy of the power source is related to the energy consumption of the device, and a power source that is drained more quickly necessitates more frequent recharging or replacement. In some instances, this may require surgery, which is highly undesirable for the patient.

### SUMMARY

According to one aspect of the present invention, a medical implant for providing electrical stimulation to a patient's tissue is provided. The medical implant comprises: at least one electrically conductive region; and a coating of self assembled monolayer (SAM) attached at least a portion of the conductive region, wherein the SAM is configured to inhibit the attachment of impedance-inducing material to the coated portion of the at least one conductive region.

According to another aspect of the present invention, a method of reducing energy consumption of a medical implant having at least one electrically conductive region for stimulating tissue of a patient is provided. The method comprises: coating at least a part of the at least one electrically conductive region with a self assembled monolayer (SAM) that inhibits attachment of impedance-inducing material to the at least one electrically conductive region.

According to a still other aspect of the present invention, a cochlear implant is provided. The cochlear implant comprises: a stimulator unit configured to generate electrical stimulation signals; an electrode array having at least one electrode contact configured to deliver the stimulation signals to a patient's tissue; and a coating of self assembled monolayer (SAM) on the electrode contact, wherein the SAM is configured to inhibit the attachment of impedance-inducing material to the electrode contact.

### BRIEF DESCRIPTION OF THE DRAWINGS

The various aspects of the present invention will now be described in detail with reference to the following figures in which:

FIG. 1 is an illustration of a medical implant having a conductive region in which embodiments of the present invention may be implemented;

FIG. 2 is an illustration of a cochlear implant in which embodiments of the present invention may be implemented;

FIG. 3 is an illustration of the electrodes of the cochlear implant FIG. 2;

FIG. 4 illustrates a stimulation bi-phasic pulse waveform as used in cochlear implant stimulation, in accordance with embodiments of the present invention;

FIG. 5 is an illustration of a medical implant with electrode contacts coated with a self assembled monolayer (SAM) in accordance with embodiments of the present invention;

FIG. 6 is a schematic illustration of an alkane thiol-based SAM on an electrode contact surface, in accordance with embodiments of the present invention;

FIG. 7 illustrates a SAM molecule structure that may be used in embodiments of the present invention;

FIG. is an illustration of an electrode contact surface contour with a SAM coating, in accordance with embodiments of the present invention;

FIG. 9 illustrates an exemplary method of coating an electrode contact with a SAM, in accordance with embodiments of the present invention;

FIG. 10 is an illustration of a medical implant coated with more than one self assembled monolayer, in accordance with embodiments of the present invention;

FIG. 11 is an illustration of a cochlear implant with electrode contacts and extra-cochlear electrode contacts, in accordance with embodiments of the present invention;

FIG. 12 illustrates part of a cochlear implant with coated electrode contacts, in accordance with embodiments of the present invention;

FIG. 13 shows a medical implant system, in accordance with embodiments of the present invention;

FIG. 14 shows a plot of impedance over time for implanted electrode contacts, in accordance with embodiments of the present invention;

FIG. 15 shows a plot of conductance vs. frequency, in accordance with embodiments of the present invention; and

FIG. 16 shows a plot of capacitance vs. frequency, in accordance with embodiments of the present invention.

### DETAILED DESCRIPTION

Aspects of the present invention are generally directed to a medical implant comprising at least one conductive region having a self-assembled monolayer (SAM) disposed thereon. The SAM is configured to inhibit the attachment of organic material to the coated portion of the at least one conductive region. Specifically, the organic material, as described below, is material that increases the impedance at the tissue/electrode interface when compared to an tissue/electrode interface that does not have the organic material. As is known in the art, a SAM is an organized layer of amphiphilic molecules in which one end of the molecule, known as the “head group” shows a special affinity for a substrate. SAMs also consist of a tail with a functional group at the terminal end.

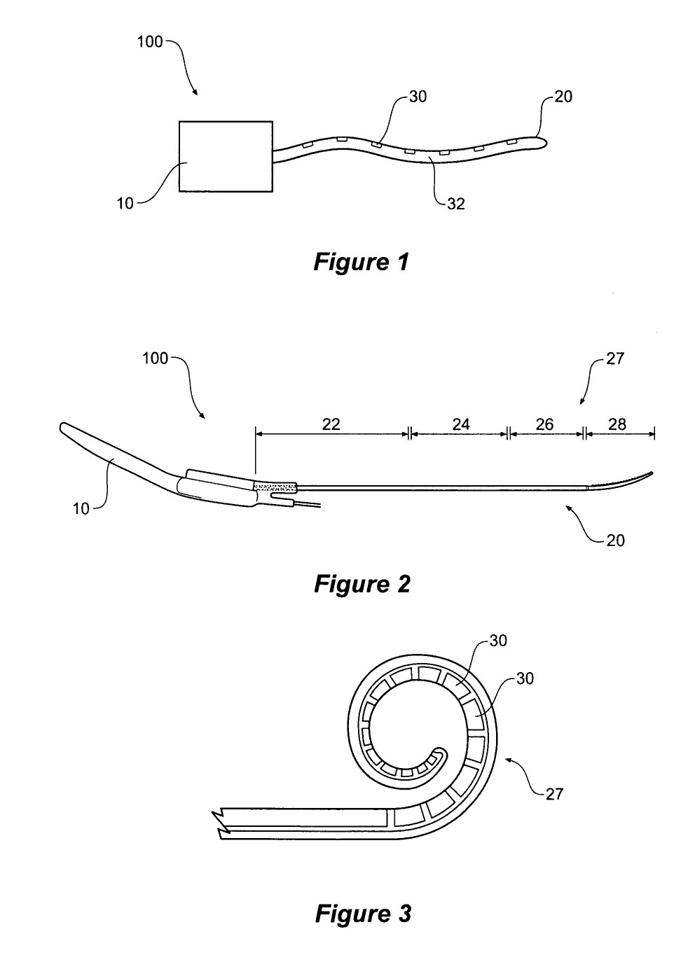

FIG. 1 is a schematic illustration of a medical implant 100 having a stimulator 10 and a lead or carrier member 20. Carrier member 20 comprises a plurality of conductive regions or electrode contacts 30 and non-conductive region 32. In the illustrative arrangement of FIG. 1, electrode contacts 30 are a plurality of discrete regions. It would be appreciated that in an alternative arrangement, a conductive region may encompass the whole or part of the lead 20.

FIGS. 2 and 3 illustrate medical implant 100 when used as a cochlear implant. In this arrangement, lead 20 is classified into four regions, the helix region 22, the transition region 24, the proximal region 26 and the intra-cochlear region 28. Proximal region 26 and intra-cochlear region 28 are collectively referred to as electrode array 27. Electrode array 27, and in particular, intra-cochlear region 28 includes a plurality of electrode contacts 30 (FIG. 3). The electrode contacts 30 are each connected to respective conducting pathways, such as wires 34 (see FIG. 5), extending through the lead 20 to the stimulator 10. As noted above, stimulator 10 generates electrical signals for each electrode contact 30 to deliver to the patient. Lead 20 may be filled with silicone to hold wires 34 in position relative to the electrode contacts 30.

An electrode contact tissue/interface interface exists between each electrode contact 30 and the electrolytes in the cochlear fluids of the patient. The term electrode contact/tissue interface is used throughout this specification to mean the interface between the stimulating electrode contact and the tissue to be stimulated. In the case of a cochlear implant, this is the interface between the electrode contact and the auditory nerve fiber tissue being stimulated.

This interface results from the difference in the conducting mediums (electron flow in the metal electrode contact 30) and to ionic flow in the cochlear fluid. With constant stimulus, a thin layer of water molecules is formed at the electrode contact 30 surfaces, and nearby is a diffused layer of hydrated ions. This forms the electric double layer.

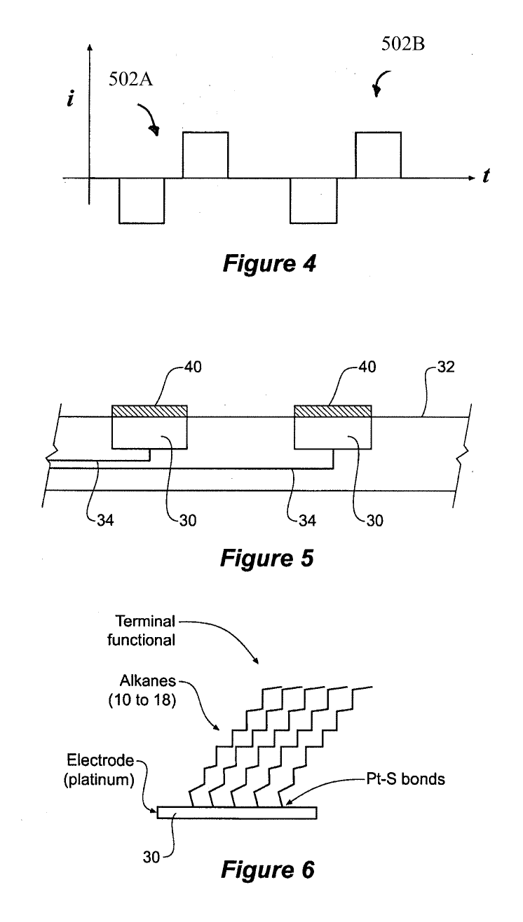

There are two primary charge transfer mechanisms for delivery of the stimulation signals to the patient. The first charge transfer mechanism is known as capacitive charge transfer and is achieved by charging the double layer that acts like a capacitance due to separation of charge. However, this alone cannot provide effective stimulation of the nerves. The other charge transfer mechanism is the faradaic reaction which involves surface chemical reactions that may be reversible or irreversible. Irreversible reactions are not desirable in medical implants because harmful chemical species can diffuse into the biological system. For that reason, a set of biphasic pulses is delivered in neural stimulation. As can be seen in FIG. 4, a biphasic pulse 502 is comprised of equal and opposite current amplitudes that balance the charge.

When a foreign object such as a medical implant is implanted into the body, various physiological reactions, such as immune response, occur in response to the presence of the foreign body. These responses may include the build up of impedance-increasing materials, such as fibrous tissue encapsulations or protein adhesions or other attachments, on the surface of the implant. Such organic materials increase the impedance at the tissue/electrode interface (i.e. the location where the electrode contacts, or is adjacent to, the tissue), when compared to a tissue/electrode interface that does not include the organic materials.

In medical implants that deliver electrical stimulation via an electrode contact, such protein adhesions, attachments, bonds or adsorptions have been found to increase the impedance of the electrode contact/tissue interface. This increased impedance can reduce the volume of tissue that can be stimulated, thus reducing the effectiveness of the implant. Furthermore, increased impedance can result in a greater drain on power sources as the required power or energy consumption of the medical implant increases to compensate for the loss in signal. In the case of an internal battery as the power source, this will necessitate more frequent changing or recharging of the battery, which in some cases may result in additional surgery for the user. In one example, the power source or battery may be located in the stimulator 10 shown in FIGS. 1 and 2).

In embodiments of the present invention, this increase in impedance, and hence increased power drain or energy consumption, is reduced by the application of a self assembled monolayer (SAM) to a portion of the surface of an electrode contact 30.

FIG. 5 illustrates one embodiment of the invention in which the electrode contacts 30 are substantially coated with a SAM 40. The electrode contacts 30 are comprised of a conductive material such as, for example, platinum, titanium, gold, palladium or other transition metals and their oxides or nitrides, carbon nano-tubes and/or conductive polymers. In one example, SAM 40 is applied to contacts to by immersing the medical implant into a thiol-containing ethanol solution. The area on which SAM 40 will attach to may be controlled by masking the implant such that only exposed regions are coated with SAM 40. As used herein, the terms “attach”, “attached” or “attachment” will be understood to include various forms of attachment, including bonding, adhesion or adsorption.

In one form, unmasked regions of the implant may be exposed to the thiol solution to enable SAM 40 to assemble and adsorb to the implant. In one embodiment, platinum electrode contacts 30 adsorbs the thiol group due to the high affinity between platinum (and other transitional metals) and the sulphur group arranged at the head of SAM 40 resulting in a covalent bond between the two. The tail of SAM 40, arranged away from the medical implant, controls the functionality of the SAM 40 and can be varied to tailor the absorbance of proteins, cells and other physiological elements. FIG. 6 shows a schematic illustration of this arrangement showing an alkane thiol-based SAM on electrode contact 30 surface.

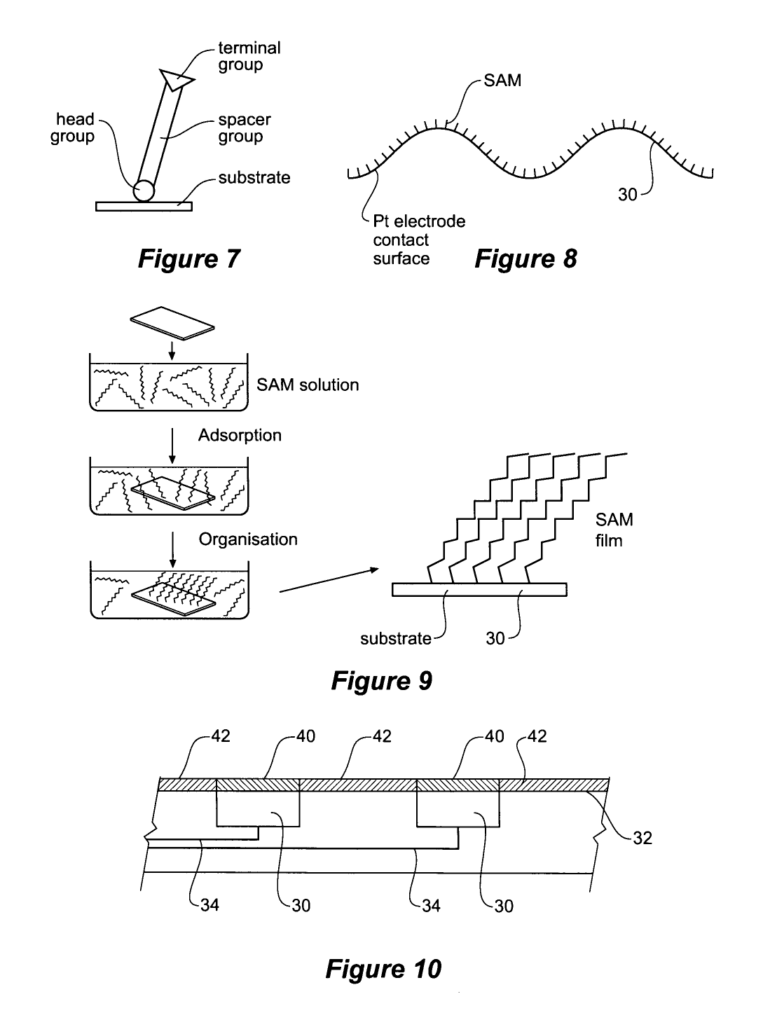

In one embodiment, SAM 40 may be represented by R—SH wherein the SH group undergoes deprotonation at the implant surface end and is covalently bound to electrode contact 30. The R group represents an alkane group, for example comprising 6 to 18 carbon atoms and terminated by a functional group, chosen depending on the purpose of the SAM binding. The terminal group of SAM 40 may be tailored to prevent or at least inhibit or alternatively, enhance tissue growth and protein attachment, adhesion, bonding or adsorption. As such, in embodiments of the invention, the terminal group of SAM 40 may comprise, but is not limited to, one or more of functional groups including COOH, CF₃, CH₃, CO₂H, NH₂, CH₂OH, CO₂CH₃ and CH₂H₄0 (ethylene oxide). FIG. 7 shows an example of such a molecule.

The majority of SAM 40 is orientated such that the tail is positioned away from the covalently bound head of the SAM. In particular, the thiol-based SAM 40 as used in the illustrated embodiment, is tightly packed and highly ordered due to electrostatic repulsive interactions between molecules in SAM 40. As such, once the medical implant is coated with SAM 40, the orientation of the molecule in addition to its terminal group preferably imparts electrostatic repulsive interactions between SAM 40 coated on the medical implant and the surrounding media, thereby deterring or inhibiting the attachment or adsorption of the impedance-inducing material, such as cells or protein, to the coated electrode contact 30. The reduction of protein adsorption (and thus impedance-inducing material) enables the medical implant to conserve charge transfer across the electrode contact/tissue interface by preventing electrode impedance fluctuations.

The thickness of SAM 40, in one form, less than 22 A, ideally results in no change to the impedance of the medical implant, and therefore no change to the efficiency of charge transfer between the implant and surrounding tissue. As such, minimal electrode impedance fluctuations may result in power saving, resulting in an increased battery life and subsequent cost savings across the life of the medical implant. This may also lead to a reduced frequency of explantation procedures for the patient.

Typically organic alkyls bearing mercapto group (thiols) can be used as SAM coatings. This is mainly due to the strong affinity that sulphur compounds have to transition metal surfaces such that the —SH group of thiol molecule undergoes deprotonation at the surface and forms a metal-sulphur covalent bond with the rest of the molecule being oriented away from the metal surface. Additionally, the interactions between the head group-substrate, end group-substrate, chain-chain, and end group-end group can be used to modify the degree of chemisorption and physisorption. These include long-chain carboxylic acids on metal oxides, organosilane species on hydroxylated glass, silicon oxides and aluminium oxides, and sulfides, disulfides, silanes, nitriles or organosulfurbased species on noble metal surfaces.

Surfaces may be engineered to either prevent or at least inhibit, or alternatively enhance tissue growth with an appropriate host response by choices of functional groups. For instance, keratinocytes have been found to grow on —COOH terminated SAMs, corneal epithelial cells tend to grow on —CF₃, —CH₃, —CO₂H, and —NH₂ terminated SAMs; and bovine aortic endothelial cells tend to grow on —CH₃, —CH₂OH, —CO₂CH₃, and —CO₂H terminal SAMs While oligo (ethylene oxide) has universal resistance to protein adsorption regardless of the nature of the protein, thus inhibiting the attachment of this impedance-inducing material to the electrode contact.

Surfaces may also be engineered to either prevent or at least inhibit, or alternatively, to enhance tissue growth with an appropriate host response by the choice of surface finishes. A roughened surface has a larger surface area compared with a smooth surface and consequently a larger conducting surface area. Additionally the smoothness of the curves can be used to affect adhesion of biofilm. For instance, macro roughness with smooth curves and overall micro smoothness plus incorporation of SAMS will provide a high conducting surface, and prevent or at least inhibit, biofilm growth and protein adhesion. FIG. 8 is an example of an electrode contact 30 having a macro rough but micro smooth surface, with a layer of SAMs.

Alternatively, 4-carboxyphenyl group could be used in place of a thiol group to bind the SAM onto the surface of an electrode contact 30. Similar to the thiol, in these embodiments the SAM would be covalently bonded and provide long term stability in terms of protein fouling at the neural stimulating surface. Various terminal functional groups can be incorporated to engineer the surface for a specific host response.

Another type of coupling mechanism for monolayers are silanes. Silanes covalently bond to oxides on surfaces (rather than directly to the metal) and may be of benefit if using substrate materials other than platinum. Once again the terminal functional groups can be tailored for specific applications.

Various methods may be used to coat electrode contact 30 with SAM 40. In one method, as shown in FIG. 9, the substrate, or electrode contact 30, is placed in a solution of SAM. This allows the molecules to adsorb, adhere, bond or otherwise attach, to the surface of the electrode contact 30 and over a period of time, the SAM molecules will organize themselves so as to provide an organic SAM film as previously described.

In one embodiment, each individual electrode contact may be coated with the SAM and then assembled into an array of electrode contacts and incorporated into a lead. One method of assembling the array and lead is described in U.S. Pat. No. 6,421,569 entitled “Cochlear Implant Electrode Array” incorporated by reference herein.

In another embodiment, an integral electrode contact “spine and comb” arrangement may be constructed as described in International Patent Application No. PCT/US2008/083794 (WO2009/065127) entitled “Electrode Array and Method of Forming an Electrode Array”, incorporated by reference herein. The electrode assembly may then be coated as described above with reference to FIG. 9 and then constructed into an electrode lead.

In a further alternative, the already-formed lead or carrier member 20 may be masked as will be understood by the person skilled in the art, to leave exposed electrode contacts for coating as previously described. In another embodiment, if a second SAM is used to coat the lead, the electrode contacts may then be masked to prevent coating with the second SAM, and the lead coated as described above.

In the illustrated embodiment of FIG. 5, SAM 40 is limited to the electrode contacts 30. However, it is also possible that a different SAM 42 may be coated on non-conductive regions 32 of the medical implant as shown in FIG. 10. In some cases, it may be desirable to promote adhesion, bonding, adsorption, binding or other attachment at some areas to provide a more secure placement of the implant within the patient. In this alternate embodiment as shown in FIG. 10, the medical implant may be coated with a first SAM 40 designed to deter protein adsorption, and subsequently coated with a second SAM 42 designed to promote protein adsorption, binding or attachment. To obtain such a coating, the medical implant may be masked to coat the implant with the first SAM 40 and later masked again to coat alternate regions with the second SAM 42 to promote protein binding in exposed regions. In this example, non-conductive regions 32 are tailored such that second SAM 42 enables protein binding to promote integration of the implant with surrounding tissue. Conductive regions 30 are coated with first SAM 40 to reduce protein fouling and reduce unnecessary charge transfer between the implant and surrounding tissue.

In further embodiments (not illustrated), the SAM may be combined with an anti-microbial coating to reduce protein fouling of implant surfaces. The anti-fouling coating may include, but is not limited to, silver ions, antibiotics, drugs, peptide coatings and poly-ethylene glycol coatings. The coating may be hydrophobic or hydrophilic.

In certain embodiments of the present invention, the conductive regions of the medical implant be biocompatible metals such as titanium, palladium, tantalum, iridium, gold and carbon-nano tubes. In these embodiments, a SAM may be applied thereto. Additionally, SAMs could also be applied over biocompatible conductive or insulative polymeric materials such as silicone and polyurethane carbon nano particle reinforced composites as well as other biocompatible materials such as alumina for blocking protein adsorption.

The benefit of SAMs on electrode surfaces to substantially prevent an increase in impedance has been discussed above. It would be appreciated that in some cases, protein blocking would also be beneficial over the implantable medical device as a whole such as the intra-cochlear portion of the implantable medical device. For instance SAMs can minimize trauma to the cochlea during explantation as a consequence of blocking protein adsorption and fibrous tissue growth on the electrode lead or carrier member. The use of the overall SAM coating of the device may be used as required depending upon circumstances such as the likelihood of explantation in a particular patient.

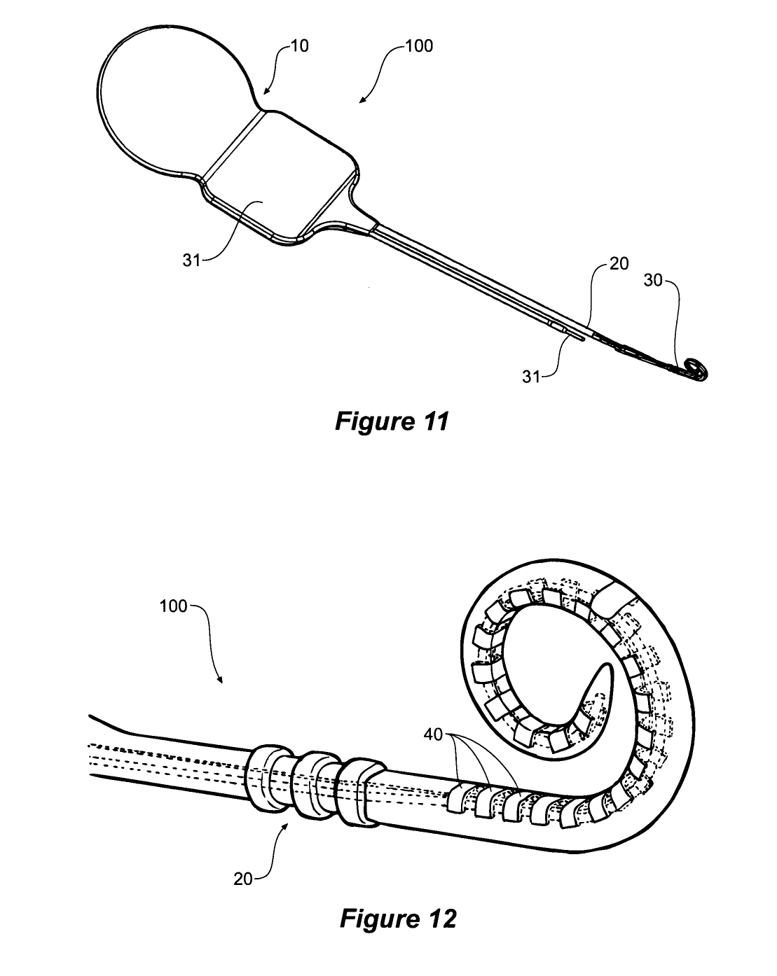

In accordance with embodiments of the present invention, SAMs may also be applied to conducting surfaces of extra cochlear electrodes (ECE) as shown in FIG. 11. FIG. 11 shows cochlear implant 100 with stimulator 10, lead 20 supporting electrode contacts 30, and extra cochlear electrodes (ECEs) 31. In this embodiment, electrode contacts 30, as well as one or more of the extra cochlear electrodes 31 are coated with SAMs. As described previously, these surfaces need not be limited to one surface morphology, but can include other high surface area to volume type structures, such as porous surfaces and meshes in order to deter protein adsorption and reduce increase in power consumption. As described above, in some embodiments, the outer surface of the rest of the implant 100 may be coated in SAMs that would promote protein or other tissue attachment.

In one embodiment of the present invention, the SAM may be tailored to block protein adsorption on a cochlear implant. In this case, the intra-cochlear portion (either the entire surface or simply a portion of one or more of the conductive electrode pads or contacts) of the medical implant may be coated with a SAM terminated by a functional group known to have resistance to protein adsorption. An example of such a group is ethylene oxide, a group that has universal resistance to protein adsorption regardless of the nature of the protein. The functional group is however not solely limited to ethylene oxide and alternate groups may be used to obtain a similar result. FIG. 12 shows a lead or carrier member 20 of a cochlear implant 100 with electrode contacts 30 coated with a SAM 40 to reduce protein adsorption to the electrode contacts.

The presence of SAMs on the electrode contact also reduces the DC bias level between electrode contacts. A high DC bias is likely due to organic residues on the electrode surface. This may also provide an effective cleaning protocol for neural stimulation applications.

Another advantage of using SAMs is that the design of the medical implant may be altered as a result. For instance, because the impedance of the electrode contact/tissue interface is reduced, the size of the electrode contacts may also be reduced. Accordingly, a greater number of electrode contacts may be incorporated into the electrode array, potentially providing a further increase in the performance of the implant.

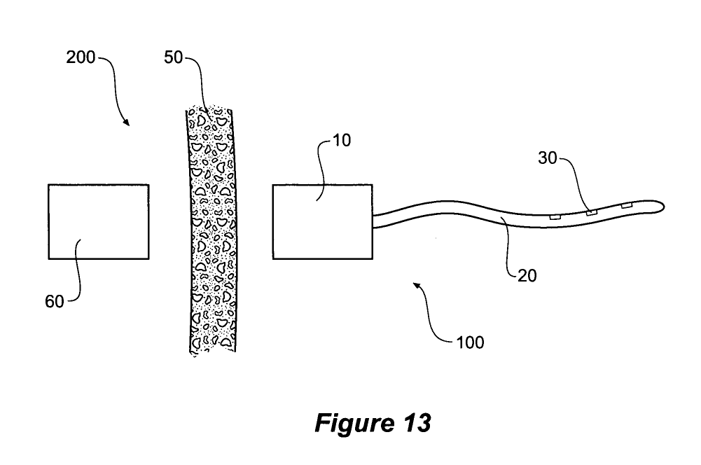

FIG. 13 shows a medical implant system 200, comprising an external component 60 and an implantable component 100 that is substantially similar to medical implant 100 of FIG. 1). An example of the medical implant system 200 is a cochlear implant system, in which the external component 60 is a sound processor that receives audio signals from the environment, and translates the audio signals into electrical signals for communication to implantable component 100. Implantable component 100 receives these electrical signals and translates these into stimulating signals for stimulating the tissue of the patient via electrode contacts, 30.

In the embodiments of FIG. 13, implantable component 100 is shown implanted into a patient beneath barrier 50. Barrier 50 may be the patient's skull and surrounding tissue. External component 60 may communicate with implant component 100 by any suitable means, including wirelessly using radio frequency (RF) signals (representing the processed input audio signals) that are generated by the processor 60 and transmitted via an antenna. The RF signals travel through skull 50 and are received by implanted stimulator 10 and converted into stimulating signals for stimulation of the patient's cochlea.

According to an aspect of the present invention, a portion of one or more of the electrically conductive regions (electrode contacts 30) of implantable component 100 may be coated with a SAM to reduce, prevent or inhibit attachment of the impedance-inducing material thereto.

According to another aspect, at least a portion of the electrically non-conductive region of implantable component 100, such as part of the casing of the stimulator 10, and/or part of lead 20 may also be coated with a second SAM that attracts the impedance-inducing material, to enhance securement of the implant within the patient.

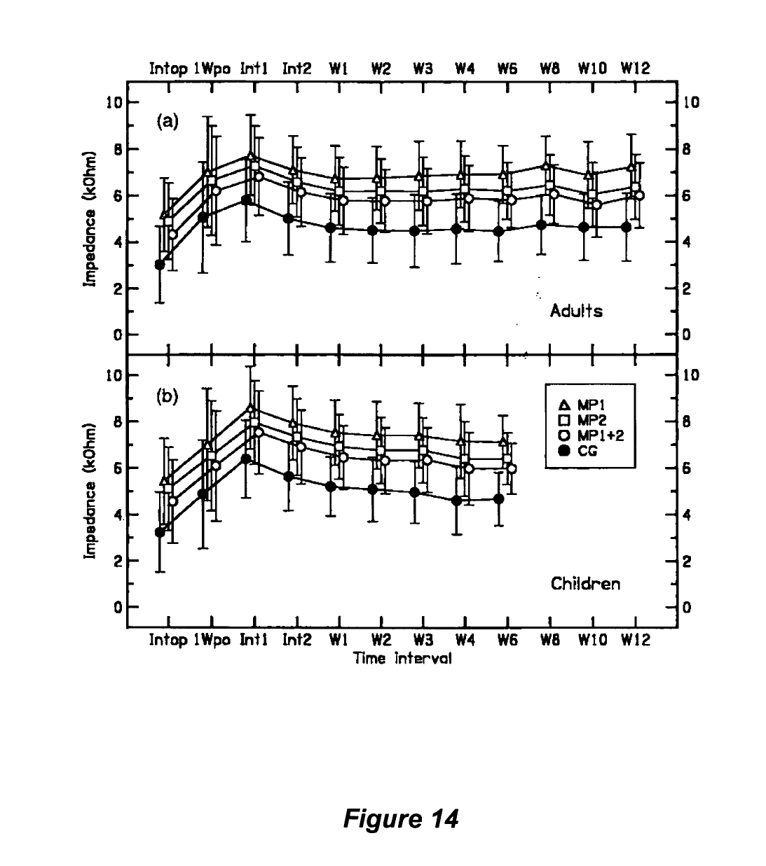

FIGS. 14, 15 and 16 show various results from experiments demonstrating the effect of the application of the SAM on an electrode contact 30.

Without any SAM coating for an electrode contact, the inter-operative impedance is typically about 1 to about 5 kOhm (in common ground (CG), and is between about 3 and about 7 kOhm when measured in monopolar modes. In post implantation the impedance is about 3 to about 6 kOhm for CG, but would be higher for MP These measurements were performed with clinical software, and the increase in impedance after implantation is due to fibrous tissue encapsulation. These measurements are shown in FIG. 14.

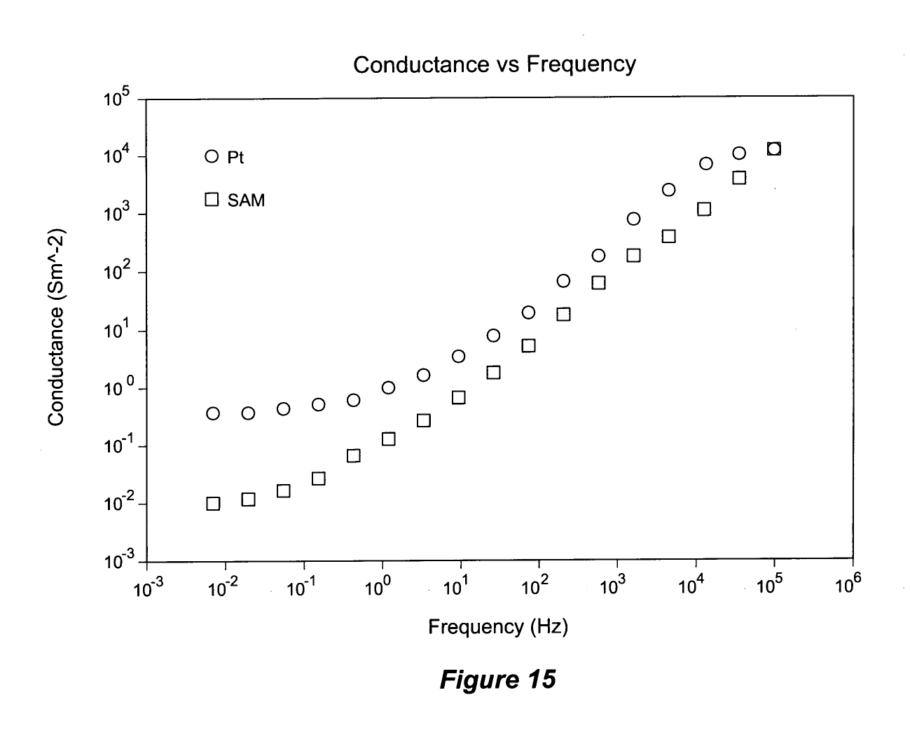

FIG. 15 shows the conductance per surface area of electrode contacts with approximately 10 times the surface area of a typical cochlear implant intra-cochlear electrode. The measurements (using electrochemical impedance spectroscopy (EIS) were performed in saline without proteins. The conductance of a SAM coated electrode contact is shown by the line represented by squares and the conductance of a bare Pt electrode contact of the same surface area is shown by the line represented by circles. From this graph the impedance for 1 kHz is about 5 kOhm for the SAM coated electrode and about 2.5 kOhms for the bare Pt electrode (given a surface area of about 2 mm²).

The above estimate was confirmed by additionally recent Custom Sound measurements in saline. These indicate about 1.93 kOhm without SAM coating and about 3.94 kOhm with the SAM coating (both values for impedance in Monopolar MP1+2), which confirms a doubling of impedance with the SAM coating. These measurements were performed with electrode contacts with a larger surface area compared to intra-cochlear electrode contacts.

### Comparability:

Test Method: EIS measurements (as conductance graph in FIG. 15) taken at 1 kHz are considered to be a good estimate for the impedance measured using custom sound.

Influence Electrolyte: It appears that impedance values taken in saline and intra-operatively are comparable. This assumption is based on measurements performed in saline and known and available intra-operative data.

Influence of electrode surface areas: The impedance increases with decreasing surface area:

The impedance was about 1.93 kOhm for a 2 mm² surface area electrode compared to about 3.55 kOhm for a 0.2 mm² electrode contact (comparable to cochlear implant intra-cochlear electrode contact). Both measurements were taken under the same conditions (in saline, at room temperature, MP1+2 mode using clinical software).

Based on the above, it is assumed that the impedance of a SAM coated cochlear implant electrode contact is about 5.5 kOhm, which is still at the lower end of the scale for the MP impedance (which is between about 4.5 and about 8.5 kOhm in adults).

The references provided above indicate that the increase in the impedance due to the SAM is much less compared with the increase in impedance post implantation.

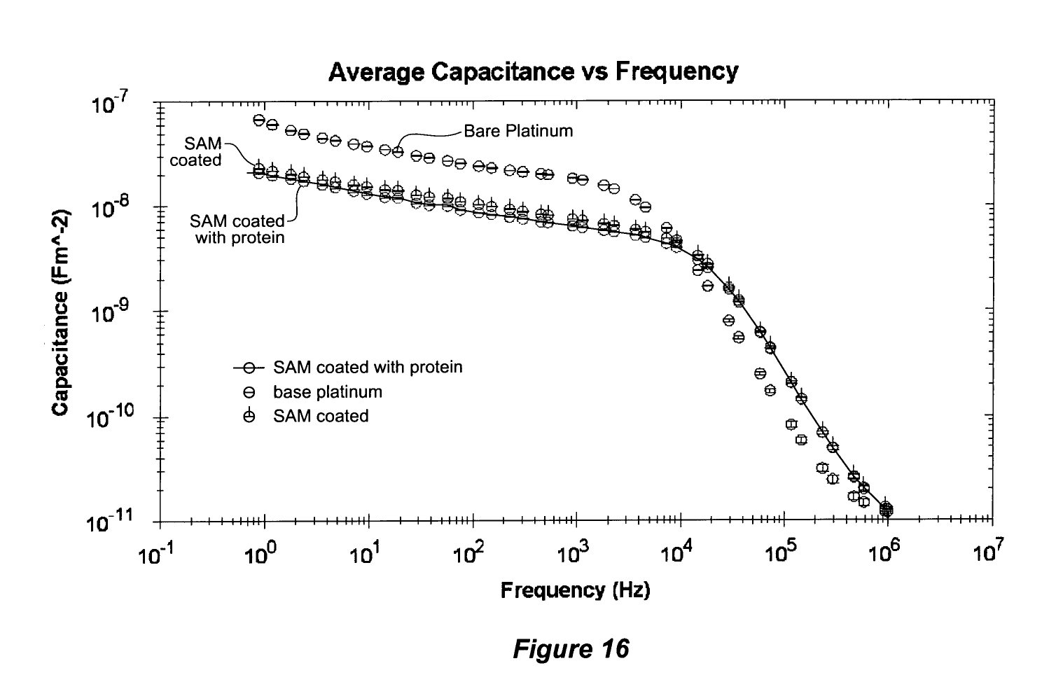

The data presented in FIG. 16 for a SAM coated electrode contact shows that there is no significant difference between capacitance vs frequency measurements for SAM coated Pt electrodes with and without protein.

The above results demonstrate that the coating repels or at least inhibits protein and cell attachment and thus inhibits the impedance-inducing material.

The various aspects of the present invention may be applied to any suitable cochlear implant, as well as any other medical implant that uses electrodes and electrical stimulation, including Auditory Brain Stem Implants, Deep Brain Stem Implants, Cardiac Pacemakers, Intraocular Retinal Prostheses, etc.

It will be understood that the term “comprise” and any of its derivatives (e.g. comprises, comprising) as used in this specification is to be taken to be inclusive of features to which it refers, and is not meant to exclude the presence of any additional features unless otherwise stated or implied.

The reference to any prior art in this specification is not, and should not be taken as, an acknowledgement of any form of suggestion that such prior art forms part of the common general knowledge.

Although an illustrative embodiment of the present invention has been described in the foregoing detailed description, it will be understood that the invention is not limited to the embodiment disclosed, but is capable of numerous rearrangements, modifications and substitutions without departing from the scope of the invention as set forth and defined by the following claims.

The above illustrations show a medical implant capable of being coated with a self assembled monolayer (SAM). It will be appreciated that the illustrations are representative only, and that the size of the monolayer in any illustration is not intended to be an accurate depiction of its true size relative to the medical implant or SAM.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All documents, patents, journal articles and other materials cited in the present application are hereby incorporated by reference.

1-19. (canceled)

20. A medical implant for providing electrical stimulation to a patient's tissue, comprising:
at least one electrically conductive region configured to interface with the tissue; and
a layer of self-assembled monolayer (SAM) coating, attached to at least a portion of the conductive region, configured to inhibit the attachment of impedance-increasing organic material to the coated portion of the at least one conductive region.

21. The implant of claim 20, further comprising at least one electrically non-conductive region adjacent the at least one conductive region, and wherein the SAM is not attached to the at least one non-conductive region.

22. The implant of claim 21, wherein at least of the non-conductive region is coated with a second, different SAM that increases the attachment of organic material to the electrically non-conductive region.

23. The implant of claim 20, wherein the SAM is an alkane thiol-based SAM.

24. The implant of claim 20, wherein the SAM comprises one or more of functional groups selected from the group consisting of: COOH, CF₃, CH₃, CO₂H, NH₂, CH₂OH, CO₂CH₃ and CH₂H₄0.

25. The implant of claim 20, wherein the surface of the at least one portion of the conductive region comprises a surface finish.

26. The implant of claim 25, wherein the surface finish comprises a roughened surface.

27. The implant of claim 20, wherein the medical implant is a cochlear implant.

28. A method of reducing energy consumption of a medical implant having at least one electrically conductive region configured to interface with tissue of a patient so as to deliver stimulation signals thereto, comprising:
coating at least one portion of the at least one conductive region with a self-assembled monolayer (SAM) that inhibits attachment of impedance-increasing organic material to the coated portion the at least one conductive region

29. The method of claim 28, wherein the medical implant comprises at least one electrically non-conductive region and the method further comprises:
coating only the at least one portion of the conductive region with the SAM.

30. The method of claim 29, further comprising:
masking the non-conductive region prior to coating the at least one portion of the conductive region.

31. The method of claim 28, further comprising:
coating only a portion of the non-conductive region with a second, different SAM that increases the attachment of organic materials to the coated non-conductive region.

32. The method of claim 31, further comprising:
masking the non-conductive region prior to coating the at least one portion of the conductive region;
coating the at least one portion of the conductive region with the SAM that inhibits attachment of the impedance-increasing organic material;
removing the mask on the non-conductive region;
masking the conductive region; and
coating the non-conductive region with a second, different SAM that increases the attachment of organic material to the coated non-conductive region.

33. The method of claim 28, wherein coating the portion of the conductive region comprises:
immersing the medical implant into a thiol-containing ethanol solution.

34. The method of claim 28, further comprising:
coating the at least one portion of the conductive region with a SAM that comprises one or more of functional groups selected from the group consisting of: COOH, CF₃, CH₃, CO₂H, NH₂, CH₂OH, CO₂CH₃ and CH₂H₄0.

35. The method of claim 28, further comprising:
surface finishing the surface of the at least one portion of the conductive region.

36. The method of claim 35, further comprising:
roughening the surface of the least one portion of the conductive region.

37. A cochlear implant, comprising:
a stimulator unit configured to generate electrical stimulation signals;
an electrode array having at least one electrode contact configured to deliver the stimulation signals to a patient's tissue; and
a coating of self assembled monolayer (SAM) on the electrode contact, wherein the SAM is configured to inhibit the attachment of impedance-increasing organic material to the electrode contact.

38. The implant of claim 37, wherein the coating substantially covers the surface of the electrode contact.

39. The implant of claim 37, wherein the electrode array comprises a non-conductive region adjacent the at least one electrode contact coated with a second, different SAM that increases the attachment of organic material to the electrically non-conductive region.
